(19) United States
(12) Patent Application Publication
(10) Pub. No.: US 2005/0194006 A1
(43) Pub. Date: Sep. 8, 2005

(54) Metered dose inhaler housing

(76) Inventor: Quyen Cao Hoang, Westminster, CA (US)

Correspondence Address: Quyen Hoang, 6174 Westbridge Ave., Westminster, CA, 92683, US

(21) Appl. No.: 10/795,170

(22) Filed: Mar. 5, 2004

(51) Int. Cl. A61M 11/00

(52) U.S. Cl. 128/200.23; 128/200.14; 128/203.12

(57) ABSTRACT

Method and apparatus for enabling a user to more easily dispense pulmonary drugs from a metered dose inhaler in a more comfortable and ergonomic manner comprising a housing with a void contained therein configured to receive a metered dose inhaler, said housing further comprising an actuation means contained therein which moves vertically to engage the drug canister of the MDI and said actuation means is initiated by the lateral motion of at least one arm positioned at a predetermined location on said housing so as to be substantially perpendicular to said housing and the drug canister of the MDI contained therein.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### BACKGROUND OF THE INVENTION

1. Field of the Invention

This invention relates generally to a device that receives and houses a metered dose inhaler and which facilitates actuation of said inhaler by the user in a more ergonomic and efficient manner as well as protects the inhaler both physically as well as from accidental discharge.

2. Discussion of Background

While the current generation of Metered Dose Inhalers (“MDI”) are functional and practical, they have several significant drawbacks. Metered Dose inhalers include a canister which contains a medicament and a propellant, a metering valve which dispenses the medicament from the canister, an actuator body that receives the canister and which forms an opening for oral inhalation, and an actuator stem which receives medicament from the canister and directs it out the opening in the actuator body. Moving the canister relative to the actuator body and actuator stem causes the metering valve to release the predetermined amount of medicament. Each metered dose inhaler is regulated by the U.S. Food and Drug Administration and each of the components is specifically designed relative to the parameters of the other components.

When the user is having difficulty breathing, the opening of the actuator body is placed in the user's mouth and then the canister is moved downwardly in the actuator so that the metering valve discharges the predetermined dose of medicament and propellant. The medicament passes through the actuator stem and then out the opening in the actuator body.

However, a problem exists with the prior art in that it requires an awkward hand and finger positioning to depress the canister to release the medicament. This problem is coupled with the need of the user to inhale concurrent with the actuation of the MDI. This actuation and breathing coordination is a common problem in the aerosol drug delivery market and can limit the optimal delivery of medicament to the user's lungs. This problem though common, is far greater in the young and elderly populations wherein manual dexterity and strength can oftentimes be quite limited. In attempts to overcome the problems associated with this manual actuation and breathing coordination problem several inhalation/breath actuated inhalers as well as assisted actuation devices have been developed. Some of the breath actuated inhalers are disclosed in U.S. Pat. Nos. 5,404,871; 5,347,998; 5,284,133; 5,217,004; 5,119,806; 5,060,643; 4,664,107. Some of the assisted actuation devices disclosed in the prior art are U.S. Pat. Nos. 6,681,763; 5,133,343; 4,649,393; 4,576,157; 3,826,413. The problems inherent in the breath actuated inhalers, are that they are expensive, mechanically complex and must be individually approved by the FDA for delivery of specific medications. The problem with the assisted actuation devices of the prior art is that they are often large and cumbersome. Said assisted actuation devices generally are no more than simple levers that are easier to grip and are located behind the MDI resulting in a larger overall footprint of the MDI as well as an unappealing appearance. The size and appearance of these assisted actuation devices draws unwanted attention as well as limits the portability of the MDI's themselves.

Thusly, what is needed is a device that can simplify the overall actuation of MDI's in a more ergonomic fashion. Furthermore, the needed device should be inexpensive, relatively compact, portable and aesthetically appealing.

### SUMMARY OF THE INVENTION

One object of this invention is to create an inhaler actuation housing device that can be accomplished by either a separate apparatus that can be used with conventional inhalers without modification of the conventional inhaler, or modification of the housing of said conventional inhaler that receives the aerosol canister.

Another object of the invention is to create a more ergonomic MDI actuation mechanism that is initiated by lateral movement of the user's finger or fingers.

Yet another object of the invention is to create a decorative and functional housing for MDI's.

Still, another object of the present invention is to create a protective housing for MDI's that physically secures said inhaler as well as protects it from accidental discharge.

Still yet another object of the invention is to create a simple and inexpensive housing for MDI's with all the benefits herein mentioned.

A key feature of the invention is that the actuation mechanism for the MDI is initiated by lateral movement of at least one protuberance extending beyond the MDI housing with said protuberance located at a predetermined point substantially perpendicular to and closer to the flat portion of the aerosol canister than to the medication discharge end of said canister.

The essential advantage of the invention therefore lies in the ability to create a simple, inexpensive housing for MDI's that can be actuated in a more comfortable and ergonomic fashion.

### BRIEF DESCRIPTION OF THE DRAWING

A more complete appreciation of the invention and many of the attendant advantages thereof will be readily obtained as the same becomes better understood by reference to the following detailed description when considered in connection with the accompanying drawings, wherein:

FIG. 1 shows a conventional MDI of the prior art;

FIG. 2 shows a profile view of an MDI of the prior art;

FIG. 3 shows a frontal view of the MDI assistive device of the present invention;

FIG. 4 shows a profile view of the MDI assistive device of the present invention;

FIG. 5 shows a frontal cutaway view of the assistive device of the present invention wherein the device is in an unactuated “rested” position;

FIG. 6 shows a frontal cutaway view of the assistive device of the present invention wherein the device is in an actuated position;

FIG. 7 shows a frontal cutaway view of the assistive device of the present invention with an alternative actuation mechanism wherein the device is in a rested position;

FIG. 8 shows a frontal cutaway view of the assistive device of the present invention with an alternative actuation mechanism wherein the device is in an actuated position.

### DESCRIPTIONS OF THE PREFERRED EMBODIMENTS

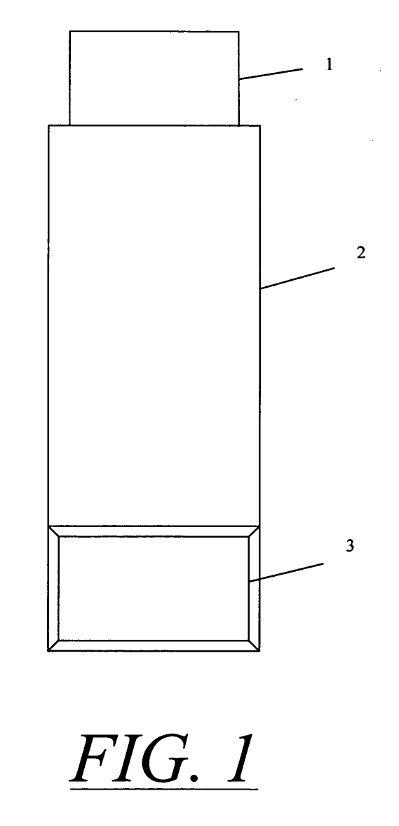

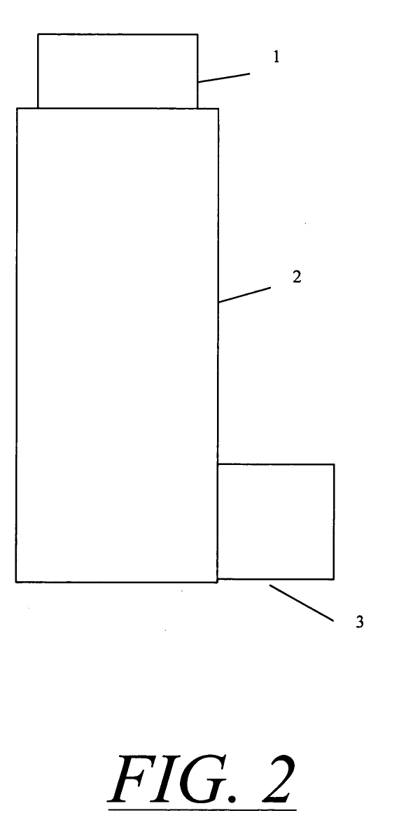

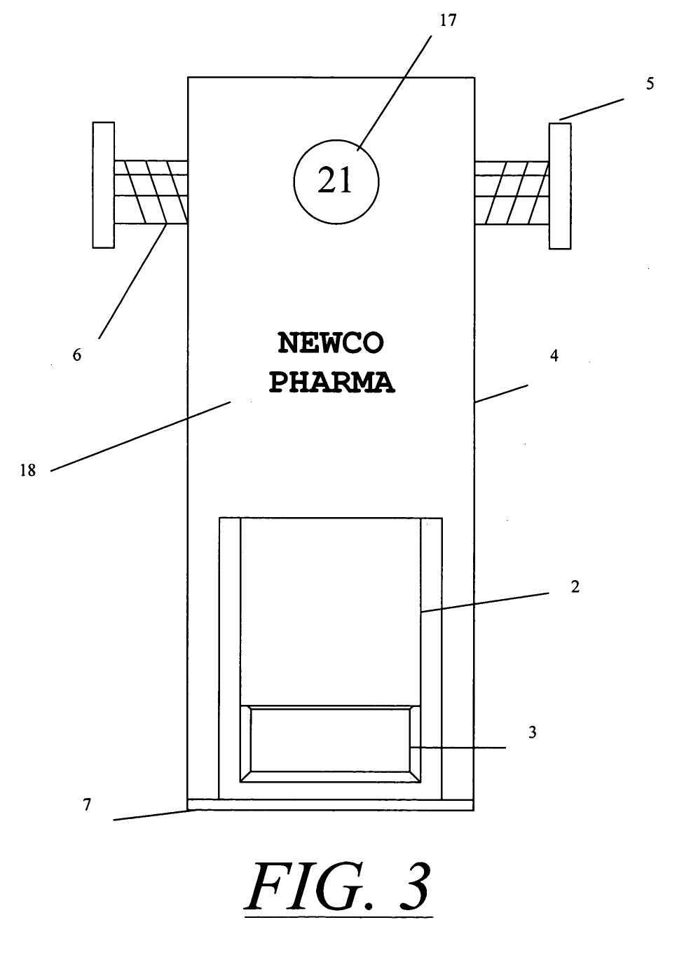

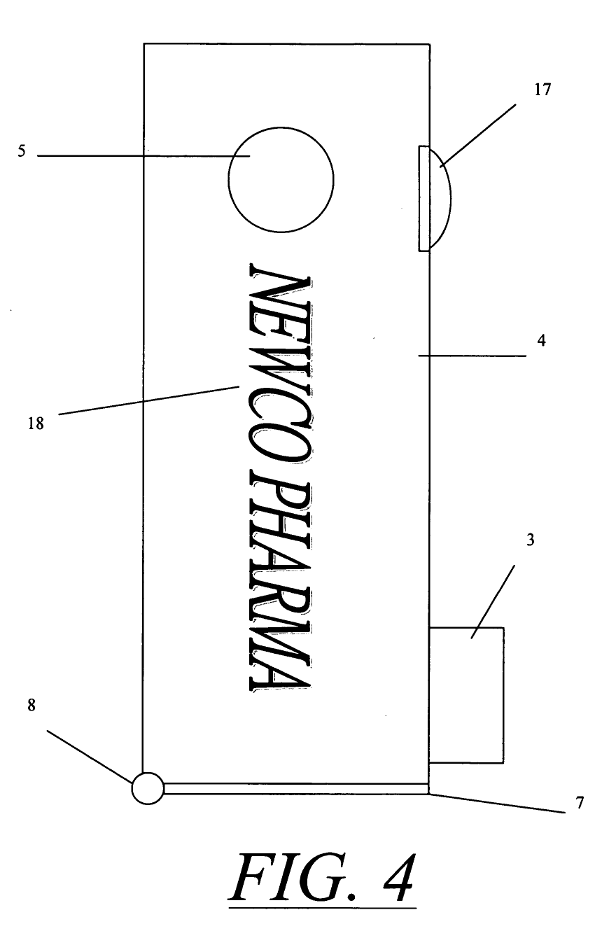

Referring now to the drawings, like reference numerals designate identical or corresponding parts throughout the several views, all elements not required for directly understanding the invention have been omitted. FIGS. 1 and 2 depicts an MDI 2 of the prior art with the drug canister 1 and mouthpiece 3 readily apparent. FIGS. 3 and 4 depicts the MDI housing device of the present invention (hereinafter, “housing or MDI assistive device”). In FIG. 3 the MDI 2 and mouth piece 3 of the prior art can be seen in it's respective position in relation to the MDI assistive device 4 of the present invention. The MDI 2 is inserted into the MDI assistive device 4 through the bottom access panel 7. The MDI 2 in the present invention is actuated by pushing on pushrods 5. Said pushrod 5 has push rod springs 6 to provide tension which after actuation brings the pushrods 5 back into a rested position. The bottom access panel 7 is pivotally attached to the main body of the MDI assistive device 4 by bottom access panel hinge 8 as seen in FIG. 4.

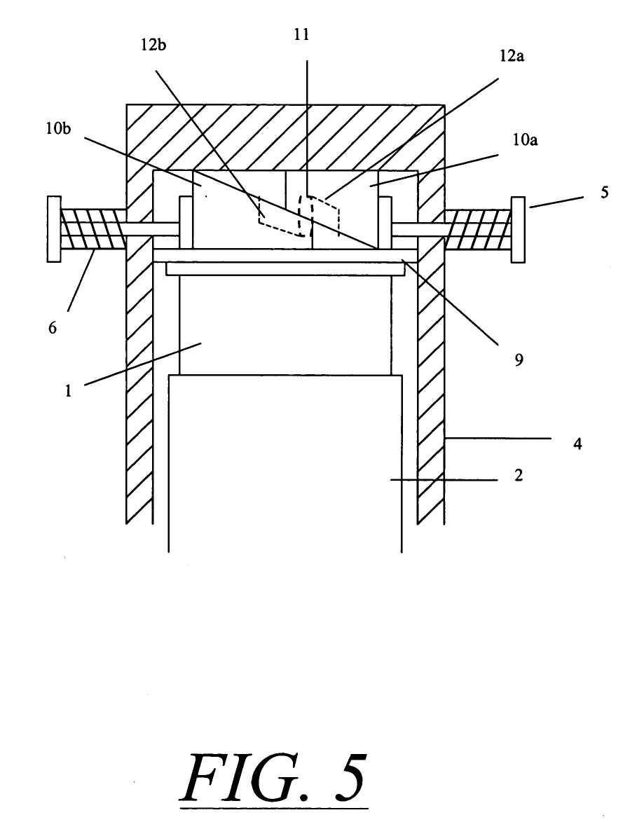

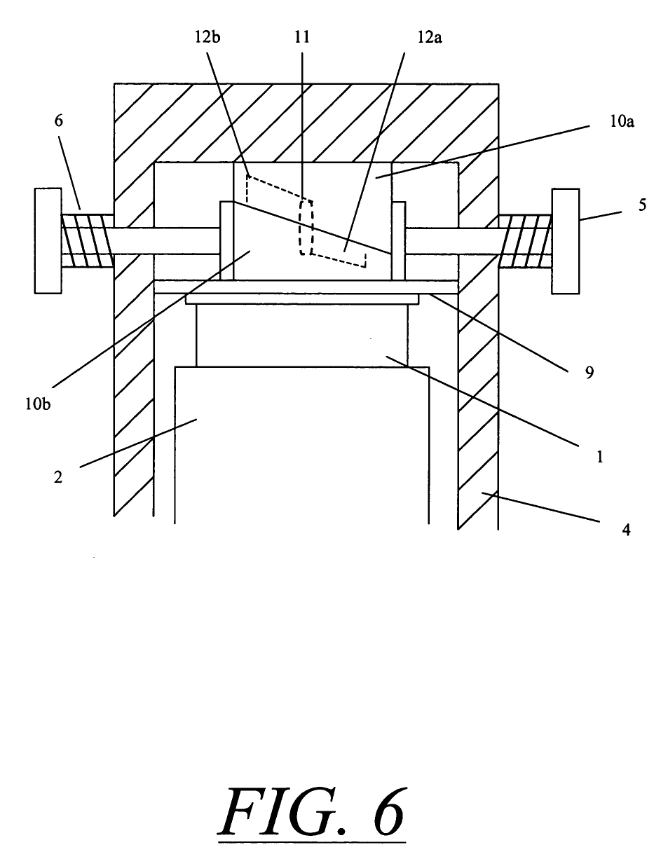

FIG. 5 shows a cutaway frontal view of the MDI assistive device 4 of the present invention in a rested position wherein the actuation mechanism is revealed as comprising two substantially triangular pressure blocks 10a and 10b connected together by guide bolt 11 which functions to guide said pressure blocks 10a and 10b into place. The guide bolt 11 is situated inside guide grooves 12a and 12b which are located on pressure blocks 10a and 10b respectively. FIG. 6 depicts a cutaway frontal view of the MDI assistive device 4 of the present invention in an actuated position wherein the push rods 5 are pushed in. As a consequence the pressure blocks 10a and 10b are pushed together to form a substantially square structure which displaces the pressure plate 9 downward. As pressure plate 9 is in contact with the top flat portion of the drug canister 1, said downward displacement of said pressure plate 9 also forces drug canister 1 downward into the MDI body 2 which results in release of the pressurized medicament inside said drug canister 1. After actuation is complete, the pushrod sprigs 6 which is under tension pushes the pushrod 5 back into a rested position. As said pushrods are connected to the respective pressure blocks 10a/10b, the pushrod springs 6 also causes said pressure blocks 10a and 10b to separate. As the pressure blocks separate, the displacement force on the pressure plate 9 is removed and the metering valve and mechanisms of drug canister 1 forces said canister back up and restored to it's rested position which in turn forces the pressure plate 9 up and back into it's respective resting position as well.

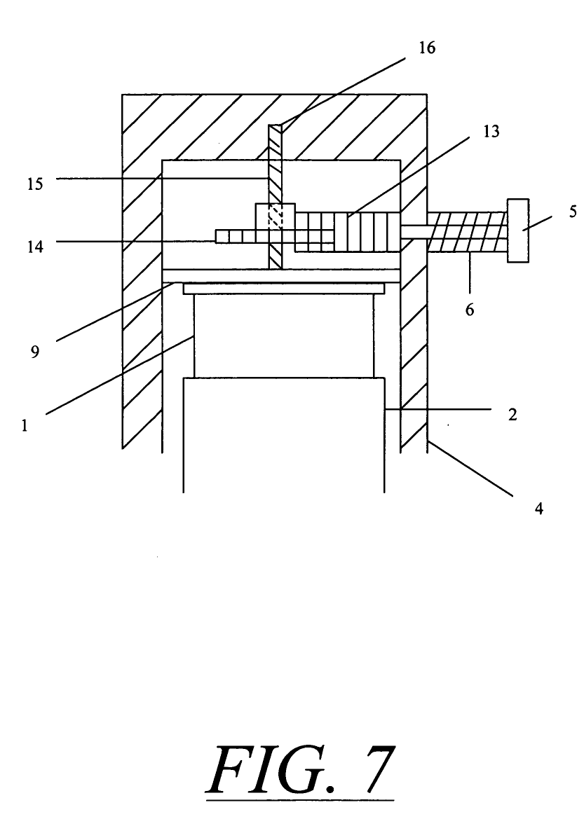

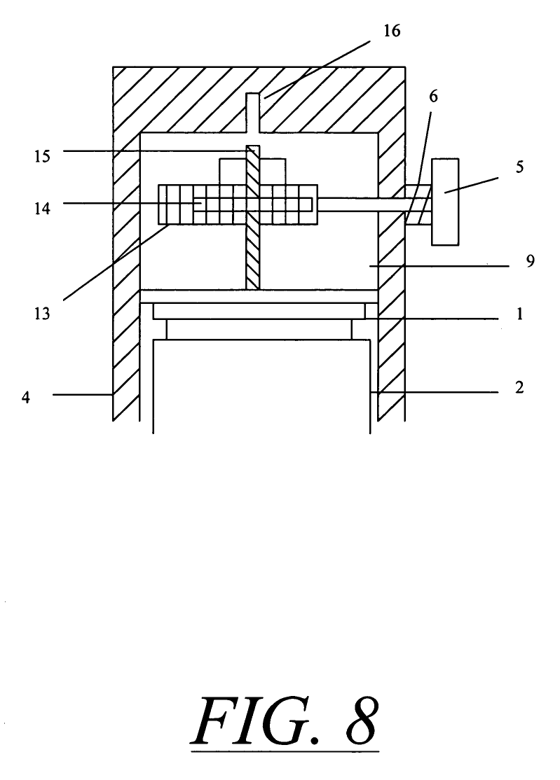

An alternative actuation mechanism of the MDI assistive device 4 of the present invention is depicted in FIG. 7 and FIG. 8. In FIG. 7 the displacement force that forces the pressure plate 9 downward to activate the drug canister 1 is the vertical movement of gear bolt 15. In this embodiment of the present invention pushrod 5 is connected to a gear rack 13 which is operatively connected to a gear wheel 14. The gear bolt 15 is operatively connected, threadedly joined and passes perpendicularly through the center of the gear wheel 14. As the pushrod 5 is pushed inwardly, the gear rack 13 moves laterally which in turn spins the gear wheel 14 around it's axis point. The internal threading of the center of the gear wheel 14 where it is threadedly joined to Gear bolt 15 forces said gear bolt to similarly rotate as said gear wheel 14 is spun. As pushrod 5 is pushed inwardly the resulting rotation of the gear wheel 14 causes the gear bolt 15 to rotate downwardly which consequently forces the pressure plate 9 downwardly and results in discharge of the pressurized medicament contained in the drug canister 1 similar to the method disclosed in the preceding paragraph.

FIG. 8 depicts the alternative embodiment of the present invention in it's actuated position wherein the gear rack 13 is moved laterally to it's furthest position rotating the gear wheel 14 so that gear bolt 15 is moved downward. In a rested position, a portion of the threaded gear bolt resides in a correspondingly threaded gear bolt recess 16. However, in the actuated position said portion of said gear bolt is driven down and substantially out of said gear bolt recess. As set forth in the disclosure of the previous embodiment above, when pressure on pushrod 5 is removed, the tension of the pushrod spring 6 forces the actuation mechanism to return to it's rested position.

From the disclosure of the present invention set forth herein, the benefit over the prior art lies mainly in the ability to actuate the MDI through lateral movement of the users fingers. In this manner, the MDI can be actuated in a more comfortable and ergonomic position without a radical departure from the shape and footprint of said MDI. Furthermore, in this manner aesthetic designs can be more easily incorporated into the MDI assistive device.

Another core benefit is the simplicity of the device which consequently translates into low manufacturing costs and a lower per unit price. In fact it is an intended benefit of the present invention to create an MDI assistive device with a low price point sufficient to make it a viable promotional product for pharmaceutical companies to give away to patients as an incentive to use their pulmonary drugs. FIG. 3 and FIG. 4, depicts the device of the present invention with a corporate mark 18 displayed prominently for promotional purposes. Furthermore, the mechanical simplicity facilitates the inclusion of a dose counting means 17 as seen in FIG. 3 and FIG. 4. wherein the dose counting means can operate as a function of the number of times the pushrod 5 has been pushed. In this manner, a relatively inexpensive mechanical or electrical or electro-mechanical dose counting means can be easily incorporated by those skilled in the art.

The invention has been described in an illustrative manner, and it is to be understood that the terminology, which has been used, is intended to be in the nature of words of description rather than of limitation. Obviously, numerous modifications and variations of the present invention are possible in light of the above teachings. It is therefore understood that within the scope of the appended claims, the invention may be practiced otherwise than as specifically described herein.

1. An accessory apparatus for use with an oral inhalation device for dispensing medication into the lungs of a user, comprising:
a. a housing with a void of a predetermined volume disposed therein configured for receiving a metered dose inhaler;
b. an actuation means having at least two component parts having a first, resting orientation and a second, actuating orientation;
c. said actuation means having at least one substantially lateral moving arm to initiate actuation, with said arm located in a predetermined position in respect to said housing so as to be substantially perpendicular to the housing and the drug canister of the MDI to be received therein;
d. the actuation means includes an interior volume displacement means that moves substantially vertically with respect to the top flat portion of the drug canister of the MDI and is situated in a predetermined location inside the void of said housing;
e. at least one energy retaining means located at a predetermined position on said actuation means to restore the actuation means from an actuated orientation back into said resting orientation;
f. at least one ingress and egress means to insert or remove the metered dose inhaler relative to the housing;
whereby a user can more easily self administer a pulmonary drug in a more comfortable and ergonomic hand position.

2. An accessory apparatus as claimed in claim 1 wherein the interior volume displacement means of the actuation means is in operative communication with the top flat portion of the MDI drug canister;

3. An accessory apparatus as claimed in claim 1 wherein the interior volume displacement means of the actuation means comprises at least two substantially solid corresponding blocks of a predetermined geometry, which when forced to slide against one another at a predetermined angle results in an object that takes up more vertical space and consequently moves a predetermined part of the actuation means vertically to engage the drug canister of the MDI to dispense a predetermined quantity of drug therefrom.

4. An accessory apparatus as claimed in claim 1 wherein the volume displacement means of the actuation means comprises at least two correspondingly geared members which when spun causes one of the said geared members to move vertically and consequently moves a predetermined part of the actuation means vertically to engage the drug canister of the MDI to dispense a predetermined quantity of drug therefrom.

5. An accessory apparatus as claimed in claim 1 wherein the energy retaining means is a spring.

6. An accessory apparatus as claimed in claim 1 wherein said housing includes a dose counting means to track the number of doses dispensed as a function of times the actuation means has been actuated.

7. An accessory apparatus as claimed in claim 1 wherein said housing includes at least one predetermined corporate brand positioned in a predetermined location on the housing.

8. A method for more easily dispensing a pulmonary drug from a metered dose inhaler in a more comfortable and ergonomic manner comprising:
(c) providing an actuation means with at least two component parts configured to actuate a Metered Dose Inhaler through vertical movement of a predetermined part of said actuation means which is in operative communication with the top flat region of the drug canister of said MDI, with the actuation means having a first, resting orientation and a second, actuating orientation;
(d) providing at least one substantially lateral moving arm to initiate the actuation means, with said arm located in a predetermined position with respect to the housing so as to be substantially perpendicular to said housing and said drug canister of the MDI;
(e) providing at least one energy retaining means located at a predetermined position on said actuation means to restore the actuation means from an actuated orientation back into said resting orientation;
(f) providing at least one ingress and egress means to insert or remove the metered dose inhaler relative to the housing;

9. The method according to claim 7, wherein providing an actuation means comprises providing an actuation means with a vertical movement caused by forcing together at least two substantially solid corresponding blocks of a predetermined geometry, which when forced to slide against one another at a predetermined angle results in an object that takes up more vertical space and consequently moves a predetermined part of the actuation means vertically to engage the drug canister of the MDI to dispense a predetermined quantity of drug therefrom.

10. The method according to claim 7, wherein providing an actuation means comprises providing an actuation means with a vertical movement caused by rotating at least two correspondingly geared members which when spun causes one of the said geared members to move vertically and consequently moves predetermined part of the actuation means vertically to engage the drug canister of the MDI to dispense a predetermined quantity of drug therefrom.

11. The method according to claim 7, wherein providing at least one energy retaining means comprises using a spring positioned at a predetermined location on said actuation means to restore the actuation means from an actuated orientation back into a resting orientation.

12. The method as claimed in claim 7, wherein providing a housing comprises providing a dose counting means to track the number of doses dispensed as a function of times the actuation means has been actuated.

13. The method as claimed in claim 7, wherein providing a housing comprises providing at least one predetermined corporate brand for promotional and branding purposes situated in a predetermined location on said housing.
